(19) United States
(12) Patent Application Publication
(10) Pub. No.: US 2012/0009163 A1
(43) Pub. Date: Jan. 12, 2012

(54) MEANS AND METHODS FOR ACTIVATING VAGUS NERVE

(75) Inventors: Daisuke SAWADA, Sagamihara-shi (JP); Shigeru FUJIWARA, Sagamihara-shi (JP)

(73) Assignee: CALPIS CO., LTD., Tokyo (JP)

(21) Appl. No.: 13/177,138

(22) Filed: Jul. 6, 2011

(30) Foreign Application Priority Data
Jul. 7, 2010 (JP) 2010-154893

(51) Int. Cl. A61K 35/74; A23L 1/30; A61P 1/00; A61K 35/00; A61P 25/00; A23K 1/16; A23L 2/52

(52) U.S. Cl. 424/93.44; 426/2; 426/61; 424/93.4; 424/93.45; 424/115

(57) ABSTRACT

An object of the present invention is to provide an effective means and method for activating vagal afferent nerve. The present invention provides a vagus nerve activator comprising bacterial cells and/or a treated product of a lactic acid bacteria as active ingredient(s).

### CROSS-REFERENCE TO RELATED APPLICATIONS

This application claims the benefit of priority from Japanese Patent Application No. 2010-154893 filed on Jul. 7, 2010, the contents of which are incorporated herein by reference.

### BACKGROUND OF THE INVENTION

1. Field of the Invention

The present invention relates to a method and means for activating vagus nerve. Specifically, the present invention relates to a vagus nerve activator containing a lactic acid bacteria. Further, the present invention relates to a method for producing a functional food product containing the vagus nerve activator, a method for activating the vagus nerve of a subject, and a pharmaceutical composition for improving cerebral blood flow.

2. Background Art

The term “vagus nerve” collectively refers to parasympathetic nerves that control thoracoabdominal organs. In particular, vagal afferent nerves are responsible for transmission of external information received by abdominal organs to the medullary nucleus tractus solitarius or the central nervous system.

Vagal cerebral blood flow changes are medically known. It is thought that cerebral blood flow changes can improve brain function. In addition, the activation of vagal afferent nerve is thought to be involved in the improvement of insomnia and quality of sleeping by reducing blood pressure and thereby inducing partial reduction in cerebral blood flow. Further, it has been known that the activation of vagal afferent nerve is associated with suppression of gastric emptying and control of secretion of PYY, CCK, and leptin, which results in suppression of a sense of satiety, food intake behaviors, and metabolism (Juhasz A, et al. Ory Hetil. 148(39): 1827-1836, 2007; Sobocki J, et al. J Physiol Pharmacol. 56 Suppl 6: 27-33, 2005; and Li Y. Curr. Med. Chem. 14(24): 2554-2563, 2007). Therefore, cerebral blood flow improvement, brain function improvement, sleep improvement, food intake suppression, and the like can be expected through the activation of vagal afferent nerve.

It has been reported that lactic acid bacteria (Lactobacillus johnsonii, Lactobacillus casei, and Lactobacillus paracasei) regulate gastric vagal efferent nerves or adrenal sympathetic efferent nerves (Katsuya Nagai et al., Journal of Intestinal Microbiology Vol. 23, No. 3, 209-216, 2009; Tanida M. et al. Neurosci Lett. 389(2):109-114, 2005; and Yamano T. et al. Life Sci. 79(20):1963-1967, 2006). However, it has not been reported that microorganisms regulate gastric or intestinal vagal afferent nerves having functions that differ completely from those of the above efferent nerves. In addition, although there is a report on sleep improvement using a lactic acid bacteria (WO 01/45722), it is assumed in this report that Lactobacillus gasseri has no effect on sleep improvement. Further, visceral fat reduction, obesity prevention, and improvement of age-related metabolism disorders using lactic acid bacteria have been reported (US 2010/0021445 A1; WO 2008/016214; JP Patent Publication No. 2009-242431 A; and Yun S I, et al. J. Appl. Microbiol. 107(5):1681-1686, 2009). Note that there are no reports on control of feed consumption using lactic acid bacteria. In addition, stress relief effects of lactic acid bacteria have been reported (Daisuke Sawada et al., General presentation titles in the Program of the 13th Annual Meeting of Intestinal Microbiology, 2009).

### SUMMARY OF THE INVENTION

An object of the present invention is to provide an effective means and method for activating vagal afferent nerve.

As a result of intensive studies conducted in order to achieve the above object, the present inventors found that lactic acid bacterial cells and fermented milk can promote the activities of gastric and intestinal vagal afferent nerve. In addition, the present inventors confirmed that intake of lactic acid bacterial cells changes cerebral blood flow and improves sleep. Based on such findings, the present inventors concluded that bacterial cells and a treated product of a lactic acid bacteria could be useful for activating vagal nerve.

Accordingly, the present invention encompasses the followings:

(1) An agent for activating vagus nerve comprising bacterial cells and/or a treated product of a lactic acid bacteria having a vagal activation activity as active ingredient(s).
(2) The agent according to (1), wherein the lactic acid bacteria is at least one bacteria belonging to a genus selected from the group consisting of the genera Lactobacillus, Bifidobacterium, Enterococcus, Leuconostoc, Streptococcus, Lactococcus, Pediococcus, and Weissella.
(3) The agent according to (2), wherein the bacteria belonging to the genus Lactobacillus is at least one member selected from the group consisting of Lactobacillus gasseri, Lactobacillus amylovorus, Lactobacillus casei, Lactobacillus paracasei, Lactobacillus zeae, Lactobacillus rhamnosus, Lactobacillus reuteri, Lactobacillus acidophilus, Lactobacillus crispatus, Lactobacillus gallinarum, Lactobacillus brevis, Lactobacillus fermentum, Lactobacillus plantarum, Lactobacillus delbrueckii subsp. bulgaricus, and Lactobacillus johnsonii.
(4) The agent according to any one of (1) to (3), wherein the lactic acid bacteria is Lactobacillus gasseri strain CP2305 (FERM BP-11331).
(5) The agent according to any one of (1) to (4), wherein the treated product of a lactic acid bacteria is a powder or suspension of the bacteria or a fermented product of the bacteria.
(6) The agent according to any one of (1) to (5), wherein the vagus nerve comprises gastric and/or intestinal vagal afferent nerves.
(7) The agent according to any one of (1) to (6), wherein the agent is orally administered.
(8) The agent according to any one of (1) to (7), wherein the agent is used for a food or drink product, feed, or pharmaceutical product.
(9) The agent according to any one of (1) to (8), wherein the agent is used for improving cerebral blood flow or suppressing food intake.
(10) A method for producing a functional food or drink product, comprising:
preparing the agent according to any one of (1) to (9); and
adding the agent to a food or drink product.
   (11) A method for activating vagus nerve in a subject, comprising:
administering bacterial cells and/or a treated product of a lactic acid bacteria having a vagal activation activity to the subject.
   (12) A pharmaceutical composition for improving cerebral blood flow, comprising bacterial cells and/or a treated product of a lactic acid bacteria having a vagal activation activity and a pharmaceutically acceptable carrier.

### EFFECTS OF THE INVENTION

The present invention provides a vagus nerve activator. The present vagus nerve activator has a vagal activation activity and thus is effective for activating cerebral blood flow, brain function improvement and sleep improvement, and suppressing food intake, for example. Thus, it can be used for medicines or health food or drink products.

### BRIEF DESCRIPTION OF THE DRAWINGS

FIG. 1 is a graph showing changes in intestinal vagal afferent nerve activity (IVNA) caused by viable lactobacillus.

FIG. 2 is a graph showing changes in an intestinal vagal afferent nerve activity (IVNA) caused by lactobacillus-fermented milk.

FIG. 3 is a graph showing changes in gastric vagal afferent nerve activity (GVNA) caused by lactobacillus-fermented milk.

FIG. 4 shows an example of brain single photon emission computed tomography (SPECT) images taken before and after intake of lactobacillus.

FIG. 5 shows an example of brain SPECT images taken before and after intake of lactobacillus.

FIG. 6 shows an example of brain SPECT images taken before and after intake of lactobacillus.

FIG. 7 is a graph showing changes in the score of quality of sleeping caused by intake of lactobacillus.

FIG. 8 is a graph showing changes in the score of latency caused by intake of lactobacillus.

FIG. 9 is a graph showing changes in the composition ratio of Bacteroides vulgatus in feces caused by intake of lactobacillus.

### DETAILED DESCRIPTION OF THE INVENTION

The present invention is described below in detail. The present invention is based on the finding that lactic acid bacteria are involved in vagal afferent nerve activation. It is considered that vagal afferent nerve activation is significant in terms of the following three points.

### (1) Improvement of Cerebral Blood Flow and Brain Function

Vagal cerebral blood flow changes are medically known. In an experiment (Example 2), human subjects were instructed to take Lactobacillus (L.) gasseri strain CP2305. Accordingly, cerebral blood flow changes (blood flow increase in the cerebral cortex, blood flow decrease in the basal ganglia, and blood flow decrease in the right eighth region of the basal ganglia) were detected. Therefore, it was presumed that intake of the strain could improve brain functions. Such cerebral blood flow changes are thought to be effective in terms of epileptic seizure risk avoidance, stroke risk reduction, cerebral aneurysm risk avoidance, behavioral moderation, and suppression of emotional stress-induced behaviors.

### (2) Improvement of Sleep

It is thought that the activation of vagal afferent nerve is involved in the improvement of insomnia and quality of sleeping by reducing blood pressure and thereby inducing partial reduction in cerebral blood flow. In fact, as a result of a questionnaire analysis from an intake test for human subjects (Example 3), improvement of quality of sleeping and latency and alleviation of sleeping disorders were confirmed, indicating that the activation of vagal afferent nerve results in sleep improvement.

### (3) Suppression of Food Intake

It has been known that the activation of vagal afferent nerve is associated with suppression of gastric emptying and control of secretion of PYY, CCK, and leptin, which results in suppression of a sense of satiety, food intake behaviors, and metabolism (Juhasz A, et al. Ory Hetil. 148(39): 1827-1836, 2007 and Sobocki J, et al. J Physiol Pharmacol. 56 Suppl 6: 27-33, 2005).

When examining the common technical knowledge in the art described above and experimental results presented herein, it can be said that lactic acid bacteria induce the activation of vagal afferent nerve so as to improve cerebral blood flow and brain function, based on which use of the bacteria for sleep improvement, food intake suppression, stress relief, and relaxation can be expected.

Therefore, the present invention relates to a vagus nerve activator (an agent for activating vagus nerve) comprising bacterial cells and/or a treated product of a lactic acid bacteria, as well as the use of the same for medicines and foods.

The lactic acid bacteria used in the present invention is a bacteria capable of producing lactic acid from saccharides via fermentation. Examples thereof include bacteria belonging to the genera Lactobacillus, Leuconostoc, Lactococcus, Pediococcus, Enterococcus, Bifidobacterium, Streptococcus, and Weissella. According to the present invention, lactic acid bacterial strains known in the art can be used as long as bacterial cells or a treated product of a lactic acid bacteria exhibit(s) a vagal activation activity. In addition, bacterial strains that have been confirmed to be safe for animals are preferable in terms of administration to/intake by animals.

Specific examples of lactic acid bacteria include bacteria belonging to the genus Lactobacillus such as Lactobacillus gasseri, Lactobacillus amylovorus, Lactobacillus casei, Lactobacillus paracasei, Lactobacillus zeae, Lactobacillus rhamnosus, Lactobacillus reuteri, Lactobacillus acidophilus, Lactobacillus crispatus, Lactobacillus gallinarum, Lactobacillus brevis, Lactobacillus fermentum, Lactobacillus plantarum, Lactobacillus delbrueckii subsp. bulgaricus, and Lactobacillus johnsonii.

In addition, other specific examples of lactic acid bacteria include bacteria belonging to the genus Bifidobacterium such as Bifidobacterium breve, Bifidobacterium longum, Bifidobacterium pseudolongum, Bifidobacterium animalis, Bifidobacterium adolescentis, Bifidobacterium bifidum, Bifidobacterium lactis, Bifidobacterium catenulatum, Bifidobacterium pseudocatenulatum, and Bifidobacterium magnum. Examples of bacteria belonging to the genus Enterococcus include Enterococcus faecalis, Enterococcus hirae, and Enterococcus faecium. Examples of bacteria belonging to the genus Streptococcus include Streptococcus thermophilus. Examples of bacteria belonging to the genus Leuconostoc include Leuconostoc mesenteroides and Leuconostoc lactis. Examples of bacteria belonging to the genus Lactococcus include Lactococcus lactis, Lactococcus plantarum, and Lactococcus raffinolactis. Examples of bacteria belonging to the genus Pediococcus include Pediococcus pentosaceus and Pediococcus damnosus. Examples of bacteria belonging to the genus Weissella include Weissella cibaria, Weissella confusa, Weissella halotolerans, Weissella hellenica, Weissella kandleri, Weissella kimchii, Weissella koreensis, Weissella minor, Weissella paramesenteroides, Weissella soli, Weissella thailandensis, and Weissella viridescens.

The term “vagal activation activity” used herein refers to an activity of promoting vagal nerve activity, and particularly, gastric and/or intestinal vagal afferent nerve activity. Vagal nerve activity can be determined based on the electrical activity of a gastric or intestinal vagal afferent nerve. Techniques for determination of such activity have been well-known in the art. For example, the method and means described in the following can be used for determination of such activity: Shen J, et al, “Olfactory stimulation with scent of lavender oil affects autonomic nerves, lipolysis and appetite in rats.” Neurosci Lett. 2005 Jul. 22-29; 383(1-2):188-93. Therefore, it is possible to determine whether or not a lactic acid bacteria or a treated product thereof has a vagal activation activity by preparing a lactic acid bacteria or a treated product thereof, administering the lactic acid bacteria or the treated product thereof to a subject such as an experimental animal, and determining changes in the electrical activity of the gastric or intestinal vagal afferent nerve of the subject.

According to the present invention, any lactic acid bacteria can be used as long as bacterial cells or a treated product thereof were evaluated as having a vagal activation activity by a method such as the above method. A preferable example of a strain of a lactic acid bacteria having a vagal activation activity is Lactobacillus gasseri strain CP2305. Lactobacillus gasseri strain CP2305 has been confirmed to have a vagal activation activity and deposited by the present applicant as FERM BP-11331 as of Sep. 11, 2007, with the International Patent Organism Depositary, the National Institute of Advanced Industrial Science and Technology (AIST) (Tsukuba Central 6, 1-1-1 Higashi, Tsukuba, Ibaraki, Japan), which is an international depository authority established under the Budapest Treaty for deposition of patent microorganisms.

Also, mutant strains or derivative strains of the above specific bacterial strains can be used in the present invention as long as they have vagal activation activities.

A lactic acid bacteria can be prepared via culture under adequate conditions using a medium conventionally used for culture of lactic acid bacteria. A natural medium or a synthetic medium can be used as a culture medium as long as it contains a carbon source, a nitrogen source, a mineral salt, and other components and it enables culture of lactic acid bacteria with efficiency. Those skilled in the art can adequately select a known medium appropriate for a bacterial strain to be used. Examples of a carbon source that can be used include lactose, glucose, sucrose, fructose, galactose, and blackstrap molasses. Examples of a nitrogen source that can be used include organic nitrogen-containing substances such as casein hydrolysate, whey protein hydrolysate, and soy protein hydrolysate. Examples of a mineral salt that can be used include phosphate, sodium, potassium, and magnesium. Examples of an appropriate medium for culture of lactic acid bacteria include an MRS liquid medium, a GAM medium, a BL medium, Briggs Liver Broth, animal milk, skim milk, and milk-derived whey. Preferably, a sterilized MRS medium can be used. Examples of a natural medium that can be used include tomato juice, carrot juice, other vegetable juice, apple juice, pineapple juice, and grape juice.

In addition, culture of lactic acid bacteria can be performed at 20° C. to 50° C., preferably 25° C. to 42° C., and more preferably approximately 37° C. under anaerobic conditions. Temperature conditions can be adjusted using a thermostatic bath, a mantle heater, a jacket, or the like. In addition, the term “anaerobic conditions” used herein refers to a low-oxygen environment in which a lactic acid bacteria can proliferate. For instance, in such environment, anaerobic conditions can be provided by using an anaerobic chamber, an anaerobic box, an airtight container or bag containing a deoxidizer, or the like, or by simply sealing a culture container in an airtight manner. The format of culture includes static culture, shake culture, and tank culture. In addition, the period of culture can be determined to be 3 hours to 96 hours. It is preferable to maintain the pH of the medium at 4.0 to 8.0 in the beginning of culture.

A specific example of preparation of lactic acid bacteria is briefly described below. For instance, when Lactobacillus gasseri strain CP2305 is used, the lactobacillus is inoculated to a medium for lactobacillus culture (e.g., an MRS liquid medium), followed by overnight culture at approximately 37° C. (for approximately 18 hours).

After culture, the obtained culture product of lactic acid bacteria can be directly used, or it may be further subjected to sterilization and crude purification via centrifugation, etc. and/or solid-liquid separation via filtration, etc. according to need. In addition, a lactic acid bacteria used in the present invention may be in the form of viable bacterial cells or dead bacterial cells and/or in the form of wet bacterial cells or dried bacterial cells.

In addition, a treated product of a lactic acid bacteria obtained by treating bacterial cells of a lactic acid bacteria may be used as long as it has the vagal activation activity of interest. Alternatively, a treated product of a lactic acid bacteria may be further subjected to treatment. Examples of such treatment are described below.

Bacterial cells and/or a treated product of a lactic acid bacteria can be prepared in the form of suspension or diluted solution by suspension or dilution in an adequate solvent. Examples of a solvent that can be used include water, physiological saline, and phosphate buffer saline (PBS).

A product can be prepared by fermenting raw milk, skim milk, or soymilk using bacterial cells and/or a treated product of a lactic acid bacteria. For instance, a lactic acid bacteria or a lactic acid bacteria subjected to optional treatment is inoculated to raw milk, skim milk, or soymilk, followed by fermentation under conditions (substantially equivalent to the above conditions for culture) known in the art. The thus obtained fermentation product can be directly used, or it may be subjected to optional treatment such as filtration, sterilization, dilution, or concentration.

A sterilized product can be prepared by sterilization treatment of bacterial cells and/or a treated product of a lactic acid bacteria. In order to subject bacterial cells and/or a treated product of a lactic acid bacteria to sterilization treatment, for example, a known technique of sterilization treatment such as filtration sterilization, radiation disinfection, superheat disinfection, or pressure disinfection can be used.

In addition, a heated product can be prepared by heat treatment of bacterial cells and/or a treated product of a lactic acid bacteria. In order to prepare such heated product, high temperature treatment (for example, at 80° C. to 150° C.) of bacterial cells and/or a treated product of a lactic acid bacteria is performed for a certain period of approximately 10 minutes to 1 hour (e.g., approximately 10 to 20 minutes).

A disrupted product or a cell-free extract can be prepared by disrupting, fracturing, comminution, size reduction, crushing, pulverization, disintegration or grinding bacterial cells and/or a treated product of a lactic acid bacteria. For instance, physical disruption (e.g., agitation or filter filtration), enzymatic lysis treatment, chemical treatment and/or autolysis induction treatment can be performed.

An extract can be obtained via extraction of bacterial cells and/or a treated product of a lactic acid bacteria with the use of an adequate aqueous or organic solvent. An extraction method is not particularly limited as long as it is an extraction method using an aqueous or organic solvent as an extraction solvent. However, an example of such method is a known method such as a method comprising immersing the lactic acid bacteria or a lactic acid bacteria subjected to optional treatment in an aqueous or organic solvent (e.g., water, methanol, or ethanol), or agitating or refluxing it in the solvent.

In addition, bacterial cells and/or a treated product of a lactic acid bacteria can be processed into the form of a powdery product (powder) or granular product via drying. Drying methods include, but not particularly limited to, spray drying, drum drying, vacuum drying, and lyophilization, which can be used alone or in combination. Upon drying, excipients may be added according to need conventionally.

Further, an ingredient or fraction having a vagal activation activity may be purified from bacterial cells and/or a treated product of a lactic acid bacteria by a known separation/purification method. Examples of such separation/purification method include: a method involving salt precipitation, or organic solvent precipitation in accordance with degrees of solubility; a method involving dialysis, ultrafiltration or gel filtration in accordance with molecular weight differences; a method involving ion-exchange chromatography in accordance with charge differences; a method involving affinity chromatography in accordance with degrees of specific binding; and a method involving hydrophobic chromatography, or reversed-phase chromatography in accordance with degrees of hydrophobicity, which can be used alone or in combinations of two or more thereof.

The above examples of treatment may be used alone or in combinations where appropriate. According to the present invention, such treated product can be used as a vagus nerve activator.

Effects of promoting vagal afferent nerve activity and the subsequent effects including cerebral blood flow improvement, brain function improvement, sleep improvement, food intake suppression can be expected through continuous intake of the above-obtained bacterial cells and/or a treated product of a lactic acid bacteria used alone or in combination with other ingredients in the form of a vagus nerve activator or formulated into a food or drink product, feed, or pharmaceutical composition. The vagus nerve activator of the present invention comprises the bacterial cells and/or a treated product of a lactic acid bacteria described above as active ingredient(s). It may contain bacterial cells and/or a treated product of a single lactic acid bacteria. Alternatively, it may contain bacterial cells and/or a treated product obtained from two or more different lactic acid bacteria. Further, it may contain a combination of two or more treated products of lactic acid bacteria treated in different ways. In addition, the vagus nerve activator of the present invention preferably contains bacterial cells of a lactic acid bacteria. This is because when it contains bacterial cells of a lactic acid bacteria, high levels of the vagal activation activity can be achieved.

Further, in addition to bacterial cells and/or a treated product of a lactic acid bacteria used as active ingredient(s), additives described below and other known brain function improving agents, sleep improving agents, and food intake suppressors can be added alone or in combinations of two or more thereof to the vagus nerve activator of the present invention if the desired activity is not inhibited.

The dosage form of the vagus nerve activator of the present invention includes, but not particularly limited to, oral formulations such as tablets, capsules, granules, powders, dust formulations, syrups, dry syrups, solutions, suspensions, and inhalers; enteral formulations such as suppositories; infusions; and parenteral injections. Of these, the vagus nerve activator of the present invention is preferably in the form of an oral formulation. In addition, a liquid formulation such as a solution or suspension may be dissolved or suspended in water or a different adequate medium immediately before use. When the vagus nerve activator of the present invention is formed into tablets or granules, coating may be performed by a known method. Further, the vagus nerve activator of the present invention may be prepared as a controlled-release formulation such as a sustained-release formulation, a delayed-release formulation, or an immediate release formulation with the use of a technique known in the art.

The vagus nerve activator in the above dosage form can be prepared according to a conventional method by formulating conventionally used additives such as excipients, disintegrators, binders, wetting agents, stabilizers, buffering agents, lubricants, preservatives, surfactants, sweeteners, flavoring agents, aromatics, acidulants, and coloring agents into the ingredients described above in accordance with the dosage form. For example, in a case in which the vagus nerve activator is prepared as a pharmaceutical composition, a pharmaceutically acceptable carrier or an additive can be incorporated into the vagus nerve activator of the present invention. Examples of such pharmaceutically acceptable carriers and additives include water, pharmaceutically acceptable organic solvents, collagen, polyvinyl alcohol, polyvinyl pyrrolidone, carboxyvinyl polymers, sodium alginate, water-soluble dextran, water-soluble dextrin, carboxymethyl starch sodium, pectin, xanthan gum, arabic gum, casein, gelatin, agar, glycerin, propylene glycol, polyethylene glycol, vaseline, paraffin, stearyl alcohol, stearic acid, human serum albumin, mannitol, sorbitol, lactose, surfactants acceptable as pharmaceutical additives, and artificial cell constructs such as liposome.

When the vagus nerve activator of the present invention contains the above additives and other agents such as a brain function improving agent, a sleep improving agent, and/or a food intake suppressor, the content of bacterial cells and/or a treated product of a lactic acid bacteria used as active ingredient(s) may depend on the dosage form thereof. For example, as the content of lactic acid bacteria, the content is generally 0.0001% to 99% by mass, preferably 0.001% to 80% by mass, and more preferably 0.001% to 75% by mass. In order to achieve intake of the desirable amount of an active ingredient, it is desirable to prepare the vagus nerve activator of the present invention in a dosage form that allows management of the daily dose. In addition, the number of bacterial cells of a lactic acid bacteria or a treated product thereof contained in the present vagus nerve activator is approximately 10⁷ cells/g to approximately 10¹² cells/g before treatment for the treated product.

The other agents such as a brain function improving agent, a sleep improving agent, and a food intake suppressor that can be added to or incorporated into the vagus nerve activator of the present invention are not limited. Examples thereof include GABA (γ-aminobutyric acid), glycin, theanine, rosemary, milk peptide, phosphatidylserine, Osmanthus fragrans, fermented Panax ginseng, activated coenzyme Q10, petit vert, Hemerocallis fulva var. sempervirens, saffron, Xylaria, Albizzia julibrissin DURAZZ, DHA (docosahexaenoic acid), EPA (eicosapentaenoic acid), isoflavone, astaxanthin, tocopherol, tocotrienol, St. John's wort, valerian, ginkgo biloba leaf extract, taurine, Relora®, anserine, carnosin, curcumin, Bacopa monnieri, vincamine, hop, α-lipoic acid, phospholipid, Chinese herbal medicines, luobuma (Apocynum venetum), Reishi (Ganoderma lucidum), glucosamine derivatives such as 1-deoxyglucosamine and 1-deoxy-N-acetyl glucosamine, chitosan oligosaccharide, chitobiose, chitotriose, limonoid, du zhong (Eucommia ulmoides) leaf glycoside, and neem extract.

Further, the vagus nerve activator of the present invention may further contain a variety of additives used for production of medicines, food or drink products, or feeds and other various substances. Examples of such substances and additives include a variety of fats and oils (e.g., plant oils such as soybean oil, corn oil, safflower oil, and olive oil, and animal fat and oil such as beef fat or sardine oil), herbal medicines (e.g., royal jelly and ginseng), amino acids (e.g., glutamine, cysteine, leucine, and arginine), polyalcohols (e.g., ethylene glycol, polyethylene glycol, propylene glycol, glycerin, and sugar alcohols such as sorbitol, erythritol, xylitol, maltitol, and mannitol), natural polymers (e.g., arabic gum, agar, water-soluble corn fibers, gelatin, xanthan gum, casein, gluten or gluten hydrolysate, lecithin, starch, and dextrin), vitamins (e.g., vitamin C and vitamin Bs), minerals (e.g., calcium, magnesium, zinc, and iron), dietary fibers (e.g., mannan, pectin, and hemicellulose), surfactants (e.g., glycerin esters of fatty acid and sorbitan esters of fatty acid), purified water, excipients (e.g., glucose, cornstarch, lactose, and dextrin), stabilizing agents, pH adjusting agents, antioxidants, sweeteners, flavoring agents, acidulants, coloring agents, and aromatics.

Further, in addition to the above active ingredients, a functional ingredient or an additive can be incorporated into the vagus nerve activator of the present invention. Examples thereof include taurine, glutathione, carnitine, creatine, coenzyme Q, glucuronic acid, glucuronolactone, Capsicum extract, ginger extract, cacao extract, guarana extract, garcinia extract, theanine, γ-aminobutyric acid, capsaicin, capsiate, a variety of organic acids, flavonoids, polyphenols, catechins, xanthine derivatives, indigestible oligosaccharides such as fructooligosaccharide, and polyvinyl pyrrolidone.

The amount of such additive can be adequately determined depending on the type of additive and the desirable amount. The content of bacterial cells and/or a treated product of a lactic acid bacteria used as active ingredient(s) may depends on the dosage form, but a desirable amount is generally 0.0001% to 99% by mass, preferably 0.001% to 80% by mass, and more preferably 0.001% to 75% by mass (if a treated product of a lactic acid bacteria is used, the content is based on the amount of a lactic acid bacteria before treatment).

Subjects of administration or intake of the vagus nerve activator of the present invention may be vertebrate animals. Specific examples thereof include mammals such as humans, primates (e.g., monkeys and chimpanzees), livestock animals (e.g., cattle, horses, pigs, and sheep), pet animals (e.g., dogs and cats), and experimental animals (e.g., mice and rats). Further, such subjects can be reptiles and birds. Particularly preferable subjects are humans for whom vagal nerve activation is expected to take place, such as humans having a risk of brain disorder, insomnia patients, and humans having stress-induced symptoms and/or obesity.

The dose of administration or intake of the vagus nerve activator of the present invention may depends on the age and body weight of a subject, an administration/intake route, the number of doses for administration/intake, and the purpose of administration (e.g., cerebral blood flow improvement, brain function improvement, sleep improvement, or food intake suppression) and other factors, and can be changed extensively at the discretion of those skilled in the art to achieve a desired effect. For example, for oral administration or intake, it is desirable to administer bacterial cells and/or a treated product of a lactic acid bacteria contained in the vagus nerve activator in an amount (in terms of the lactic acid bacteria amount) of generally approximately 10⁶ cells to 10¹² cells and preferably approximately 10⁷ cells to 10¹¹ cells per kilogram of body weight. The content of bacterial cells and/or a treated product of a lactic acid bacteria is not particularly limited and can be adequately adjusted in accordance with the degree of ease of production, and the preferable daily dose, for example. The vagus nerve activator of the present invention is safe and thus it is also possible to further increase the amount to be administered. The daily dose may be administered in a single dose, or it may be divided into several doses. In addition, the frequency of administration or intake is not particularly limited, and it can be adequately selected depending on various conditions such as an administration/intake route, the age and body weight of a subject, and desired effects (e.g., cerebral blood flow improvement, brain function improvement, sleep improvement, and food intake suppression).

The administration/intake route of the vagus nerve activator of the present invention is not particularly limited, and includes oral administration/intake, and parenteral administration (e.g., intrarectal, subcutaneous, intramuscular, or intravenous administration). Particularly preferably, the vagus nerve activator of the present invention is orally administered or taken.

The vagus nerve activator of the present invention has a vagal activation activity and thus exhibits effects of cerebral blood flow improvement, brain function improvement, sleep improvement, and food intake suppression. Specifically, the vagus nerve activator of the present invention improves cerebral blood flow (e.g., blood flow increase in the cerebral cortex, suppression of the blood flow in the basal ganglia, or suppression of the blood flow in the right eighth region of the basal ganglia), thereby improving brain functions, which is effective in terms of epileptic seizure risk avoidance, stroke risk reduction, cerebral aneurysm risk avoidance, moderation of behaviors, and/or suppression of emotional stress-induced behaviors. In addition, the vagus nerve activator of the present invention is effective for improving sleep; that is to say, for improving quality of sleeping or latency, or alleviating sleep disorders. Further, the vagus nerve activator of the present invention is effective for suppressing food intake behaviors and metabolism.

The vagus nerve activator of the present invention may be used in combination with a different medicine or a different treatment or prevention method. A different pharmaceutical and the vagus nerve activator of the present invention may be formulated into a single formulation. Alternatively, they may be formulated into separate formulations so as to be administered simultaneously or at intervals.

As described above, the vagus nerve activator of the present invention can be used as a pharmaceutical composition for improving cerebral blood flow, brain function, or sleep, or suppressing food intake.

In addition, the vagus nerve activator of the present invention is safe and thus is easily used for long-term continuous intake. Therefore, the vagus nerve activator of the present invention can also be added in food or drink products or feeds. The vagus nerve activator of the present invention has a vagal activation activity, and it contains a lactic acid bacteria that has been conventionally used for meals and thus is safe. Further, even when it is added to a variety of food or drink products, it does not inhibit the flavor of a food or drink product itself. Thus, it can be continuously taken by adding it to a different food or drink product with the expectation of promotion of vagal afferent nerve activity.

The food or drink product of the present invention contains the vagus nerve activator described above. The food or drink product of the present invention also includes beverages. Examples of the food or drink product containing the vagus nerve activator of the present invention include all food or drink products into which the above vagus nerve activator can be incorporated, for example, food or drink products such as health food or drink products, functional food or drink products, and food or drink products for specified health use having vagal activation activities for health promotion.

Functional food or drink products are particularly preferable as food or drink products containing the vagus nerve activator of the present invention. The “functional food or drink product” of the present invention means a food or drink product having predetermined functionality for organisms and encompasses, for example, so-called general health food or drink products such as food or drink products with health claims including food for specified health use (including qualified FOSHU [food for specified health use]) and food or drink products with nutrient function claims, food or drink products for special dietary uses, nutritional supplements, health supplements, supplements (e.g., those having a variety of dosage forms such as tablets, coated tablets, sugar-coated tablets, capsules, and liquid agents), and beauty food or drink products (e.g., diet food or drink products). The functional food or drink products of the present invention also encompass health food or drink products to which Health claim based on the food standards of Codex (Joint FAO/WHO Food Standards Programme) is applied.

Specific examples of food or drink products include health food or drink products and nutritional supplements in preparation forms such as liquid diets (e.g., tube enteral nutritional supplements), tablet candies, tablets, chewable tablets, dust formulations, powders, capsules, granules, and tonic drinks; tea beverages such as green tea, oolong tea, and black tea; drinks or beverages such as soft drinks, jelly beverages, isotonic beverages, milk beverages, carbonated beverages, vegetable beverages, juice beverages, fermented vegetable beverages, fermented juice beverages, fermented milk beverages (e.g., yogurt), lactic acid bacteria beverages, milk beverages (e.g., coffee milk and fruit milk), beverages containing drink powders, cocoa beverages, milk, and purified water; spreads such as butter, jam, dried seasoning products, and margarine; mayonnaise; shortening; custard; dressings; bread; boiled rice; noodles; pasta; miso soup; tofu; yogurt; soup or sauce; and sweets (e.g., biscuits and cookies, chocolate, candies, cake, ice cream, chewing gum, and tablets).

The food or drink product of the present invention can be produced according to a conventional method by adding other food materials used for production of the above food or drink products, various nutrients, various vitamins, minerals, dietary fibers, and various additives (e.g., taste components, sweeteners, acidulants such as organic acids, stabilizers, and flavors), in addition to the above vagus nerve activator.

For the food or drink product of the present invention, those skilled in the art can adequately determine the amount of the vagus nerve activator formulated in consideration of the form of the food or drink product and the taste or texture that are required. Usually, an appropriate amount of the vagus nerve activator is generally 0.0001% to 99% by mass, preferably 0.001% to 80% by mass, and more preferably 0.001% to 75% by mass in total of bacterial cells and/or a treated product of a lactic acid bacteria in the vagus nerve activator to be added (based on the content of bacterial cells). The vagus nerve activator of the present invention is safe, and thus the amount thereof in a food or drink product can be further increased. In order to achieve consumption of the desirable amount of the vagus nerve activator, it is desirable to prepare the vagus nerve activator in a dosage form that allows management of the daily amount. As described above, the food or drink product of the present invention can be consumed in a form that allows management of the desirable amount of the vagus nerve activator of the present invention. Accordingly, a method using the food or drink product for improving cerebral blood flow, brain function, or sleep, or suppressing food intake can be provided.

The vagus nerve activator of the present invention may be incorporated into a food or drink product by an arbitrary appropriate method available by those skilled in the art. For example, the vagus nerve activator of the present invention can be prepared in a liquid, gel, solid, powder, or granule form and then incorporated into a food or drink product. Alternatively, the vagus nerve activator of the present invention may be mixed or dissolved directly into raw materials for a food or drink product. The vagus nerve activator of the present invention may be applied to, coated onto, infiltrated into, or sprayed onto a food or drink product. The vagus nerve activator of the present invention may be dispersed uniformly or distributed unevenly in a food or drink product. A capsule containing the vagus nerve activator of the present invention may be prepared. An edible film or food coating agent may be wrapped around the vagus nerve activator of the present invention. Alternatively, the vagus nerve activator may be prepared into a form such as a tablet after the addition of an appropriate excipient and others. The food or drink product comprising the vagus nerve activator of the present invention may further be processed. Such a processed product is also encompassed within the scope of the present invention.

In the production of the food or drink product of the present invention, a variety of additives as routinely used in food or drink products may be employed. Examples of the additives include, but not limited to, color formers (e.g., sodium nitrite), coloring agents (e.g., gardenia pigments and Red 102), flavors (e.g., orange flavors), sweeteners (e.g., stevia and aspartame), preservatives (e.g., sodium acetate and sorbic acid), emulsifiers (e.g., sodium chondroitin sulfate and propylene glycol esters of fatty acid), antioxidants (e.g., disodium EDTA and vitamin C), pH adjusters (e.g., citric acid), chemical seasonings (e.g., sodium inosinate), thickeners (e.g., xanthan gum), swelling agents (e.g., calcium carbonate), antifoaming agents (e.g., calcium phosphate), binding agents (e.g., sodium polyphosphate), nutrition-enriching agents (e.g., calcium-enriching agents and vitamin A), and excipients (e.g., water-soluble dextrin). Functional raw materials such as Panax ginseng extracts, Acanthopanax senticosus Harms extracts, eucalyptus extracts, or du zhong tea extracts may further be added.

As described above, the food or drink product of the present invention has a vagal activation activity. Therefore, it has effects of cerebral blood flow improvement, brain function improvement, sleep improvement, and food intake suppression. In addition, it is safe, and thus there is no concern about side effects. Further, the vagus nerve activator of the present invention has a favorable flavor. Therefore, even when it is added to a variety of food or drink products, it does not inhibit the flavor of a food or drink product itself. Accordingly, the obtained food or drink product can be easily used for long-term continuous intake with the expectation of long-term promotion of vagal afferent nerve activity.

Further, the vagus nerve activator of the present invention can be formulated not only into food or drink products for humans but also into feeds for animals such as livestock (e.g., cattle and pigs), racehorses, and pets (e.g., dogs and cats). Feeds are substantially equivalent to food or drink products except that they are given to non-human subjects. Therefore, the above descriptions of food or drink products can be applied mutatis mutandis to feeds.

### EXAMPLES

The present invention is hereafter described in greater detail with reference to the following examples, although the present invention is not limited thereto.

### Reference Example 1

Lactobacillus gasseri strain CP2305 was cultured in an MRS liquid medium and then lyophilized to obtain a powder. Thus, a sample was prepared (10⁷ cfu/ml).

Lactobacillus gasseri strain CP2305 was cultured in a liquid medium containing skim milk and a yeast extract and then lyophilized to obtain a powder. Thus, lactobacillus-fermented milk was prepared (10⁷ cfu/ml).

Lactobacillus gasseri strain CP2305 was cultured in a liquid medium containing a sugar source, a meat extract, protein hydrolysate, a yeast extract, salts, and others and then lyophilized to obtain a powder. Thus, a lactobacillus powder was prepared.

The lactobacillus strain CP2305 was cultured in a liquid medium containing skim milk and a yeast extract and then a sugar, salts, a flavor, and others were added thereinto. Thus, a sterilized lactobacillus beverage was prepared.

### Example 1

In this Example, the influence of a lactic acid bacteria on the gastric and intestinal vagal afferent nerve activity was examined.

Male Wistar rats (approximately 9 weeks old) were anesthetized. The intestinal or gastric vagal afferent nerve of each rat was lifted with an electrode to determine the electrical activity of the nerve.

A sample of the lactobacillus strain CP2305 or fermented milk thereof (1 ml) (10⁷ cfu) prepared as described in Reference Example 1 was orally administered to the rats. Then, changes in the electrical activity of the intestinal or gastric vagal afferent nerve were electrophysiologically determined. As a control, rats were received water orally and subjected to determination in the manner described above.

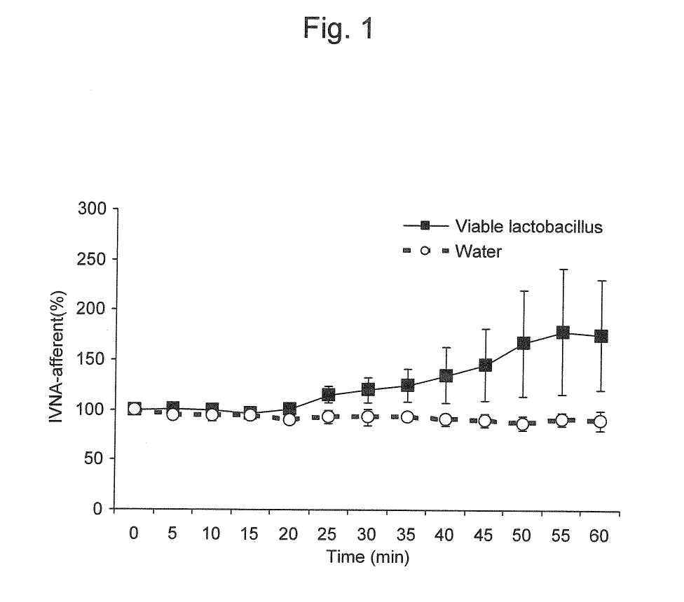

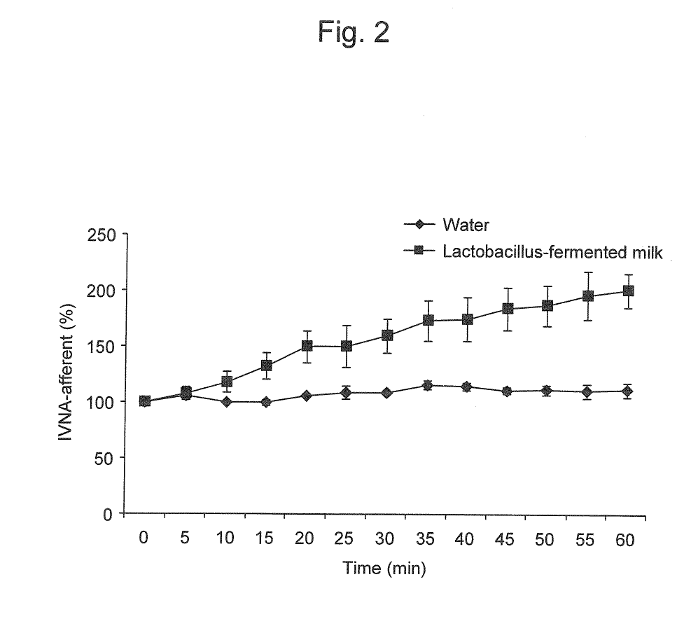

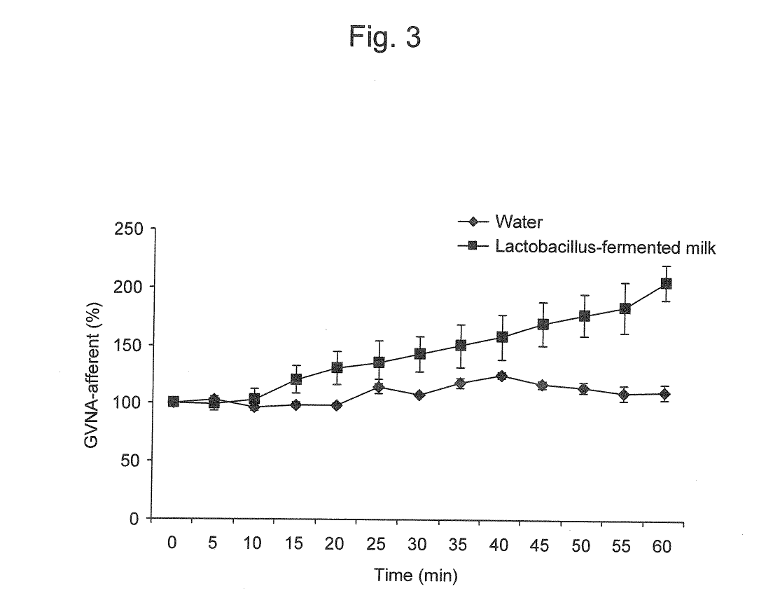

The above experimental results are shown in FIGS. 1 to 3. FIGS. 1 and 2 show changes in the intestinal vagal afferent nerve activity (IVNA) caused by viable lactobacillus and those caused by lactobacillus-fermented milk, respectively. FIG. 3 shows changes in the gastric vagal afferent nerve activity (GVNA) caused by lactobacillus-fermented milk. These results indicate that viable lactobacillus and lactobacillus-fermented milk can activate the gastric and intestinal vagal afferent nerves.

### Example 2

In this Example, the influence of lactic acid bacteria on brain functions was examined.

Eight healthy subjects were instructed to take 0.2 g of a powder of viable bacterial cells of the lactobacillus strain CP2305 prepared as described in Reference Example 1 once daily for 3 weeks. Before and after intake, single photon emission computed tomography (SPECT) was performed to obtain cerebral blood flow images.

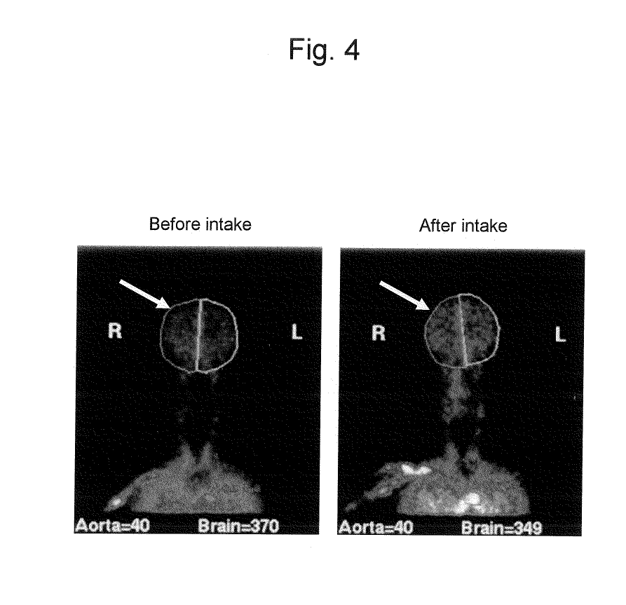

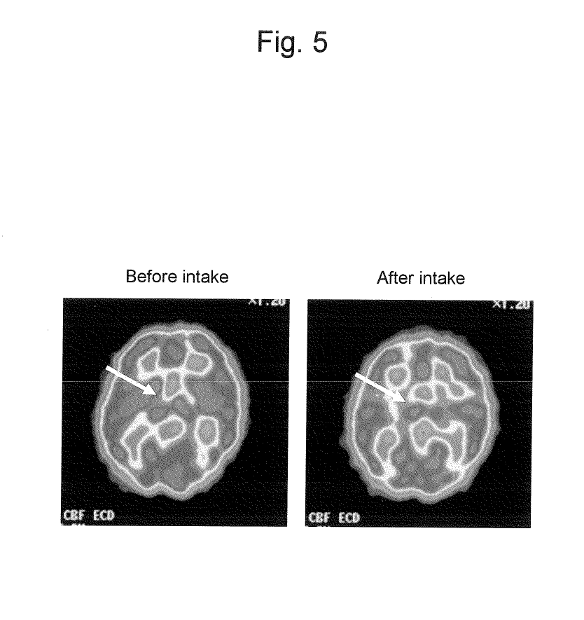

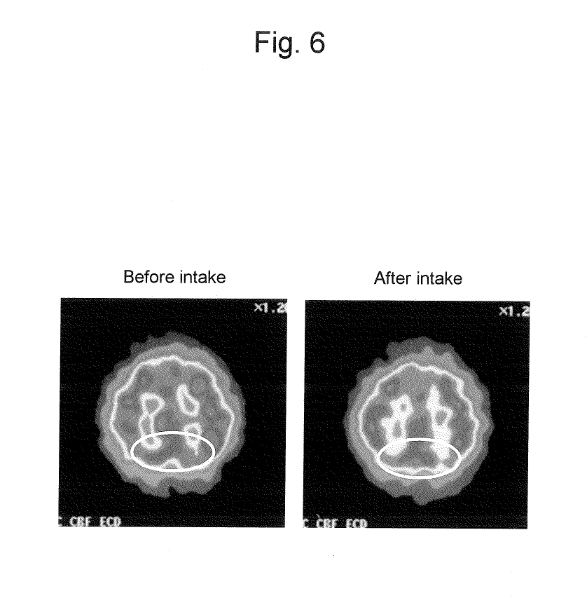

Representative cerebral blood flow images are shown in FIGS. 4 to 6. FIG. 4 shows the blood flow in the cerebral cortex (white arrow). FIG. 5 shows the blood flow in the basal ganglia (white arrow). FIG. 6 shows the blood flow in the right eighth region of the basal ganglia (white circle). Intake of the strain CP2305 caused blood flow increase in the cerebral cortex (FIG. 4), suppression of blood flow in the basal ganglia (FIG. 5), and suppression of blood flow in the right eighth region of the basal ganglia (FIG. 6). These results indicate that there is a possibility that a lactic acid bacteria would improve cerebral blood flow and brain function.

### Example 3

In this Example, the influence of a lactic acid bacteria on sleeping was examined.

Thirty two healthy subjects under stress were divided into two groups. One group was instructed to take 200 ml of a lactobacillus beverage (subjected to heat sterilization) produced with the lactobacillus strain CP2305 for 35 consecutive days while the other group was instructed to take a placebo beverage (non-fermented milk prepared as a modified product with lactic acid) during the same period. Health conditions checked before and after intake were evaluated. Before and after intake, the subjects were instructed to answer a questionnaire regarding quality of sleeping, and then the obtained scores were compared. In addition, the composition ratio of Bacteroides vulgatus (a harmful intestinal bacterium) was examined by analyzing microbiota in feces.

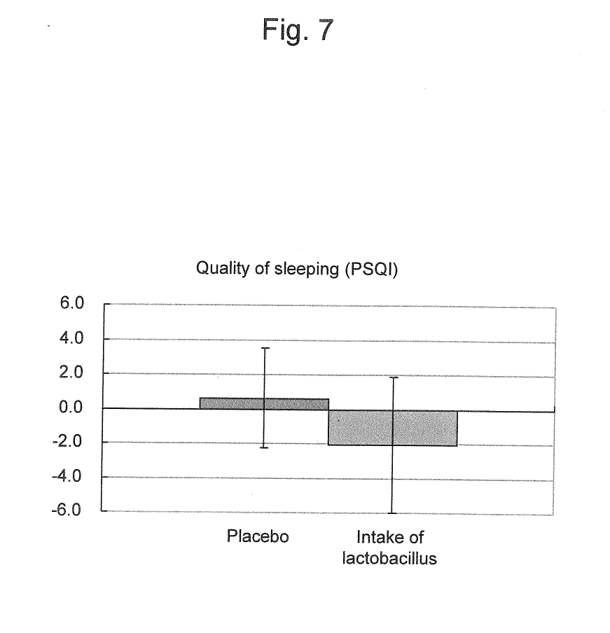

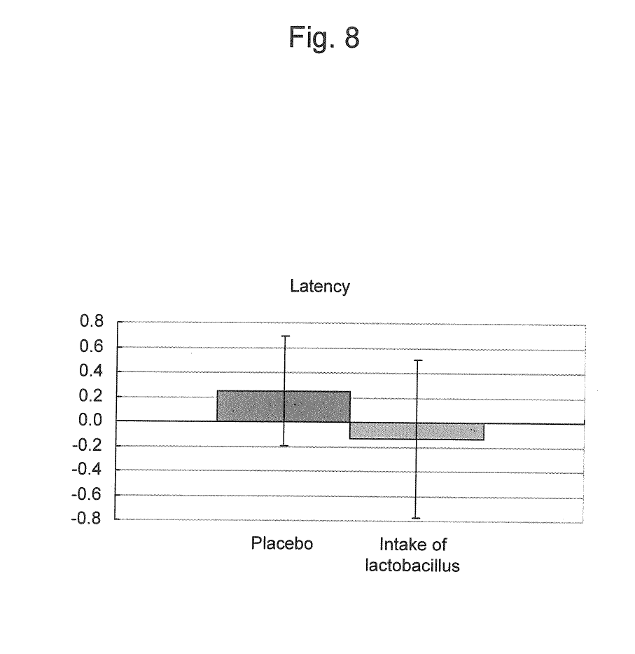

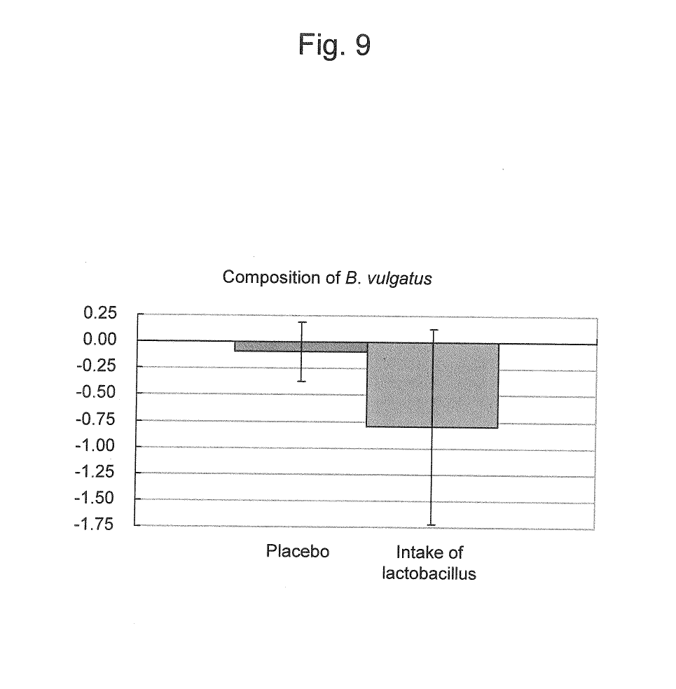

The results are shown in FIGS. 7 to 9. FIG. 7 shows score changes in quality of sleeping. FIG. 8 shows score changes in latency. FIG. 9 shows changes in the composition ratio of Bacteroides vulgatus in feces. Based on these results, it was found that intake of CP2305 strain improves quality of sleeping and sleep induction (FIGS. 7 and 8) and suppresses the composition ratio of intestinal Bacteroides vulgatus (FIG. 9). These results indicates that intake of a lactic acid bacteria is effective for sleep improvement.

The present invention provides a vagus nerve activator. The present vagus nerve activator has a vagal activation activity and thus is effective for improving cerebral blood flow, brain function or sleep, or suppressing food intake, for example. Therefore, it can be used for medicines and health food or drink products. Accordingly, the present invention is useful in the fields relating to pharmaceutical products, food or drink products, and livestock.

All publications, patents, and patent applications cited herein are incorporated herein by reference in their entirety.

What is claimed is:

1. A method for activating vagus nerve in a subject, comprising:
administering bacterial cells and/or a treated product of a lactic acid bacteria having a vagal activation activity to the subject.

2. The method according to claim 1, wherein the lactic acid bacteria is at least one bacteria belonging to a genus selected from the group consisting of the genera Lactobacillus, Bifidobacterium, Enterococcus, Leuconostoc, Streptococcus, Lactococcus, Pediococcus, and Weissella.

3. The method according to claim 2, wherein the bacteria belonging to the genus Lactobacillus is at least one member selected from the group consisting of Lactobacillus gasseri, Lactobacillus amylovorus, Lactobacillus casei, Lactobacillus paracasei, Lactobacillus zeae, Lactobacillus rhamnosus, Lactobacillus reuteri, Lactobacillus acidophilus, Lactobacillus crispatus, Lactobacillus gallinarum, Lactobacillus brevis, Lactobacillus fermentum, Lactobacillus plantarum, Lactobacillus delbrueckii subsp. bulgaricus, and Lactobacillus johnsonii.

4. The method according to claim 1, wherein the lactic acid bacteria is Lactobacillus gasseri strain CP2305 (FERM BP-11331).

5. The method according to claim 1, wherein the treated product of a lactic acid bacteria is a powder or suspension of the bacteria or a fermented product of the bacteria.

6. The method according to claim 1, wherein the vagus nerve comprises gastric and/or intestinal vagal afferent nerves.

7. The method according to claim 1, wherein the bacterial cells and/or treated product of the lactic acid bacteria are orally administered.

8. The method according to claim 1, wherein the bacterial cells and/or treated product of the lactic acid bacteria are in the form of a food or drink product, feed, or pharmaceutical product.

9. A method for improving cerebral blood flow or suppressing food intake in a subject, comprising:
administering bacterial cells and/or a treated product of a lactic acid bacteria having a vagal activation activity to the subject.

10. A method for producing a pharmaceutical composition, a functional food or drink product, comprising:
preparing bacterial cells and/or a treated product of a lactic acid bacteria having a vagal activation activity; and
adding the bacterial cells and/or treated product of the lactic acid bacteria to a pharmaceutical composition, a food or drink product.
